(19) United States
(12) Patent Application Publication
(10) Pub. No.: US 2004/0158318 A1
(43) Pub. Date: Aug. 12, 2004

(54) Surface-modified bioactive suppressant surgical implants

(75) Inventor: Louis R. Matson, El Dorado Hills, CA (US)

Correspondence Address: MICHAEL BEST & FRIEDRICH, LLP, ONE SOUTH PINCKNEY STREET, P O BOX 1806, MADISON, WI, 53701

(73) Assignee: Surgica Corporation.

(21) Appl. No.: 10/764,716

(22) Filed: Jan. 26, 2004

Related U.S. Application Data
(63) Continuation of application No. 10/038,068, filed on Oct. 17, 2001.
Application No. 10/038,068 is a continuation of application No. 09/517,932, filed on Mar. 3, 2000, now abandoned.
(60) Provisional application No. 60/122,892, filed on Mar. 5, 1999.

(51) Int. Cl. A61F 2/06

(52) U.S. Cl. 623/1.46; 427/2.25; 623/1.49

(57) ABSTRACT

Disclosed are methods of reducing undesired tissue growth adjacent to, upon, or within surgical implants. Surgical implants, especially endoprosthetic implants, are rendered bioactively suppressant by the presence of a galvanically releasable silver component and a metal more noble than silver such as gold, platinum, or rhodium, deposited on a surface of the implant, when contacted with a physiologic electrolyte, which is generally deposited as a surface coating, which provides in vivo a sustained release of silver ions in a concentration effective to reduce undesired tissue growth, but insufficient to cause serious damage to connective tissue.

### CROSS-REFERENCE TO RELATED APPLICATIONS

This application claims priority to U.S. Provisional Application Serial No. 60/122,892, filed Mar. 5, 1999.

### STATEMENT REGARDING FEDERALLY SPONSORED RESEARCH OR DEVELOPMENT

Not applicable.

### BACKGROUND OF THE INVENTION

The present invention relates to endoprosthetic implants for the human or animal body and provides methods of rendering such implants bioactively suppressant (i.e., the implants suppress undesired tissue growth adjacent to, upon or within the implant).

With few exceptions, it has been the established practice for many years to select materials for the manufacture of endoprosthetic implants that induce minimal tissue response and effects, and which possess adequate mechanical properties for the intended function or application of the implant. Structural materials that do not corrode in vivo or cause bone reabsorption are sought for use in endoprosthetic implants.

Early endoprosthetic implants were made from common metals and their alloys. Materials used for endoprosthetic implants have progressed from surgical stainless steel used in early endoprosthetics to cobalt chromium molybdenum alloys and titanium and titanium alloys, which are commonly used today.

Other materials used in endoprosthetic implants include ceramic and carbon-based materials and certain synthetic plastics materials, such as ultrahigh molecular weight polyethylene, some forms of nylon, polymethylmethacrylate, and silicone elastomers. None of these materials is entirely bioinert (i.e. bioinactive) in all circumstances. An important consideration in endoprosthetic design is the identification and development of more fully inert materials to minimize or eliminate adverse in vivo interactions or effects. The search for such materials is ongoing.

In the early years of implant surgery, silver was employed in the manufacture of some endoprosthetic implants. For example, silver wire, silver plates and silver-plated screws were used in bone repair surgery, and tracheotomy tubes were silver plated. However, by the mid-1930's, silver and silver plated implants were no longer commonly used. Silver is generally considered to be unacceptable as an implant material, particularly for orthopedic implants, because it has poor mechanical properties and it can cause connective tissue reaction and excessive subperiosteal bone growth.

Surgical implants rendered antimicrobial by the presence of a bioerodible metallic silver component on or within the implant were disclosed in U.S. Pat. No. 4,615,705, which is incorporated by reference herein. In order for the metallic silver to be effective in reducing microbial growth, the silver must be activated to provide a sustained release of silver ions in vivo in a concentration sufficient to provide a localized antimicrobial effect without causing connective tissue damage. The silver is activated or made bioerodible by heating the silver component or by treating the silver component with hydrogen peroxide, which results in the formation of silver oxides.

Silver-impregnated catheters have been used to reduce the risk of infection associated with central venous catheters. However, experience with tunneled catheter placement suggests that positioning a silver impregnated Dacron cuff often results in unusually weak subcutaneous anchorage. Hemmerlein et al. hypothesized that a silver-mediated process causes an undesired decrease in fibroblast ingrowth into the cuff, thereby resulting in weak anchorage of the catheter (Radiology 204:363-367, 1997). This hypothesis was supported by tests designed to evaluate the ability of the silver cuff material to inhibit the growth of cultured fibroblasts in vitro (Radiology 204:363-367, 1997).

In many surgical implant applications it is desirable to inhibit or minimize the ingrowth of fibroblasts and other tissues to maintain optimum functionality. Surgical stents, shunts, and indwelling catheters are good examples of implants in which selectively controlled or minimal tissue ingrowth is needed.

What is needed in the art is a method for rendering an endoprosthetic implant bioactively suppressant.

### BRIEF DESCRIPTION OF THE SEVERAL VIEWS OF THE DRAWINGS

Not applicable.

### BRIEF SUMMARY OF THE INVENTION

The present invention provides a method of reducing undesired postimplantation tissue growth in a human or animal subject following endoprosthetic implantation, the method comprising selecting a suitable endoprosthetic implant, wherein the implant comprises an implant structure formed of a substantially bioinert structural material, galvanically releasable silver component, and at least one metal more noble than silver selected from the group consisting of gold, platinum, and rhodium, wherein the silver is deposited on at least a portion of the surface of the implant structure, and wherein the more noble metal is deposited on at least a portion of the surface of the implant structure; and implanting the implant into the subject under such conditions that a physiological electrolyte contacts the silver and more noble metal.

In another aspect, the invention provides a method of rendering bioactively suppressant an endoprosthetic implant comprising an implant structure formed of a substantially bioinert structural material providing mechanical integrity to the implant, a galvanically releasable component, and at least one metal more noble than silver selected from the group consisting of gold, platinum, and rhodium, wherein the silver and more noble metal is deposited on at least a portion of the implant in or on the implant structure, comprising contacting the silver and more noble metal with an electrolyte.

A further aspect of the invention is a method of forming an endoprosthetic implant capable of being bioactively suppressant when contacted with a physiological electrolyte, the method comprising providing an implant structure formed of a substantially bioinert structural material, depositing galvanically releasable silver on at least a portion of the surface of the implant structure, and depositing at least one metal more noble than silver selected from the group consisting of gold, platinum, and rhodium on at least a portion of the surface of the implant structure.

In a preferred embodiment, the galvanically releasable silver and at least one metal more noble than silver selected from the group consisting of gold, platinum, and rhodium are provided as an alloy that is deposited on or in at least a portion of a bioinert structural implant material to form a bioactive suppressant endoprosthetic implant. Following suitable placement of the implant in the body, the silver is activated by the galvanic effect that occurs when a physiologic electrolyte such as blood, cerebrospinal fluid, urine, aqueous humor, synovial fluid, or other body fluid contacts the silver and more noble metal of the implant.

It is an advantage that by the method of the invention, it is possible to deliver an in vivo, sustained release of silver ions in a concentration effective to provide a localized bioactively suppressant effect, while causing minimal connective tissue damage. In particular, the methods provide a prophylactic treatment that reduces the risk of undesirable excessive postoperative tissue growth.

The composite implant formed by the method of the invention is self-contained, and it requires no external energy source (e.g., electrical current) to generate the suppressant activity. In a preferred embodiment, the activity is provided by the galvanic release of silver ions. The silver ions thus produced are not accompanied by the release of excessively irritating or toxic anions, such as those generated by freely dissociable silver salts that have been used as antimicrobial agents (e.g., silver nitrate).

The method is adaptable to a variety of implant types and implantation sites, and can be regularly employed in implant surgery. Depending on the desired and specific effect needed for any particular surgical implant, one may achieve the desired characteristics for a particular implant by selecting suitable parameters. For example, when the silver and a metal more noble than silver are supplied as an alloy, implant properties could be designed by selecting the relative percentage of metals in an alloy (e.g., silver and gold). Other parameters that may be varied include the absolute amount of silver in the implant structure, suitable implant structural material, and the site or sites at which the silver and the more noble metal is deposited on the implant.

Other features and advantages of the present invention will become apparent upon review of the specification.

### DETAILED DESCRIPTION

Endoprosthetics are used in a variety of applications in the treatment of various disease states to promote human health. However, implantation of endoprosthetics in the body is associated with certain risks, including the infiltration and growth of fibroblasts or other undesired tissues on the implant. Colonization of an implant by fibroblasts or other cells can cause implant failure. The present invention provides methods for inhibiting or minimizing undesired tissue growth adjacent to, upon, or within surgical implants.

Silver salts or especially silver oxides used in implant devices for control of bacterial infections are reported to cause undesired tissue irritation. Recently, heart valves coated with Silizone coating, a silver component included to reduce the risk of endocarditis associated with implantation, were recalled because Silizone is suspected of placing patients receiving the valves at increased risk of paravalvular leakage (St. Jude Press Release, Jan. 24, 2000), possibly due to tissue damage caused by silver oxides.

The present invention uses silver components in implants to reduce undesired tissue growth without substantially irritating or killing surrounding tissue. Reduction of fibroblast proliferation may be achieved by releasing silver ions over time in a more controlled manner than the methods used to prevent bacterial infection (i.e., release of silver ions from silver salts or oxides formed on metallic silver). The silver ions generated by the method of the invention have a localized effect, and act to reduce undesired tissue growth at or near the surface of the implant where such growth is undesired.

The present invention provides a method for the sustained and controlled release of silver ions from an implant. The release of silver ions is caused by the galvanic action that results when the silver and gold, platinum, or rhodium in the implant are contacted with an electrolyte. The in vivo production of silver ions obviates the need for activating the silver by other means (e.g., exposure to hydrogen peroxide or heating) prior to implantation.

In a preferred embodiment, silver is provided as an alloy with a more noble metal, such as gold, platinum, or rhodium, and deposited on at least a portion of the endoprosthetic device by any suitable deposition means. The endoprosthetic is suitably implanted in a human or other animal, and the alloy is contacted with a physiologic electrolyte (e.g., blood or other body fluid). Upon in vivo exposure to a physiological electrolyte, a galvanic effect is produced, causing the release of silver ions. In an alternative embodiment, the alloy is treated by electro-explosion to produce a powder that with a particle size of 50 nanometers or greater, which may be mixed with a semipermeable polymer capable of allowing passage of electrolytes, and deposited onto the surface of the implant.

Alternatively, the silver and the more noble metal may be distributed in a particulate form in a suitable polymer coating permeable to electrolytes is deposited on at least a portion of the implant structure. It is expected that suitable molar ratios of the silver and more noble metal present in the polymer coating will be comparable to that observed for silver alloy deposited on the implant surface. One factor that may affect the suitable ratios include the presence or absence of an additional layer on the polymer layer. The distance between the silver and more noble metal particles will also affect the rate of ion release, with higher rates obtainable with greater separation between particles.

In another embodiment, the more noble metal and the silver are separately deposited at discrete locations on the implant. It is also envisioned that an implant may be plated with gold, platinum, or rhodium and, using techniques known to one skilled in the art, the silver may be deposited to a selective region or regions of the implant at which tissue growth may be particularly undesired or problematic.

Depending on the requirements of a particular application, the implant can be designed to release ions at various rates over longer or shorter periods of time. In the case of an implant comprising a silver alloy, this may be accomplished by judicious selection of the ratio of silver to gold, platinum, or rhodium. An alloy having a relatively high ratio of silver to gold or platinum will have a higher rate of silver ion release compare with an alloy having a lower ratio.

The amount of silver or silver-containing material applied to the implant structure will determine the relative length of time over which ions are released. In general, the greater the amount of silver that an implant has, the greater the period of time over which silver ions will be released. When the supply of silver is exhausted, the implant no longer is biologically active. The gold, platinum, or rhodium will remain as an inert, biocompatible layer.

The location of the silver or silver alloy on or within the implant may be selected with regard to the structure or intended application of the implant. In the case in which the silver or silver alloy is coated onto the implant, the coating may cover the entire implant surface or only a select part or parts of the implant structure. Optionally, the silver or silver alloy may be strategically deposited over specific regions of the implant where protection against undesired tissue growth is particularly important. Alternatively, zonal tissue growth suppressant effect may be achieved by applying a corrosion mask to selected areas to prevent galvanic activation and subsequent release of silver ions. Any biocompatible coating that is capable of providing dielectric isolation may be used as a corrosion mask, including, for example, Teflon or silicone. Another suitable masking material is Parylene (Paritronix, Inc.). Parylene is a biocompatible material that prevents electrolyte from passing, thereby preventing the galvanic release of silver ions.

Suitable means for depositing silver, a more noble metal, or silver alloy include, but are not limited to, electroplating, ion implantation, or ion beam associated deposition). Preferably, the plated endoprosthetic is subjected to gas plasma treatment in argon gas to clean the implant prior to so as to reduce the risk of excessive tissue damage caused by residual silver oxides that may have formed on the surface of the implant.

Optionally, the implant wall may be surface-treated before the silver, a more noble metal, or silver alloy is applied. The surface-treatment may include the interposition of an additional layer between the wall and the silver, a more noble metal, or silver alloy to increase adhesion to the implant. A thin, semi-permeable top coating of polymer, for example a polyvinyl pyrrolidone, may be applied to control release of silver ions, to improve surface smoothness, or to prevent deactivation of the silver by interfering body fluid constituents. Additionally, the polymer coating could also contain pharmaceutical agents to enhance the bioactively suppressant effect or biocompatability.

Conveniently, the silver component is constituted by a surface coating on at least part of the implant structure. However, the component can be constituted in other ways, for example as a deposit in one or more cavities provided in the permanent implant structure or as a permeate in a porous substrate in or on the permanent implant structure.

The method of the invention is particularly suited to endoprosthetic implants having a permanent structural integrity including, but not limited to, orthopedic, ophthalmic, urological, gastroenterological, neurological, vascular, and cardiovascular implants. As used herein, the term “endoprosthetic implant” may include the entire implant, parts thereof or fixing means therefor. In particular, the term includes, for example, orthopedic pins, plates, screws, artificial joints, glaucoma implants, urological stents, esophageal stents, neurological shunts, vascular shunts, indwelling catheters, anti-adhesion barriers, sutures, and cardiovascular stents.

An “implant surface” may include an exterior or interior surface. For example, cathers have an outer wall with a surface, and an inner wall with a surface that defines the lumen of the catheter.

By an implant that is “bioactively suppressant” it is meant an implant that has an activity that reduces or prevents undesired tissue growth on the implant.

A material that is “substantially bioinert” as used herein is a material that is sufficiently nonreactive under the conditions in which the implant is used in vivo that the mechanical integrity of the implant is retained despite erosion of the metallic silver. The implant can be made of any structural material which is substantially bioinert, but it is expected that materials such as titanium or titanium alloy, or cobalt chrome molybdenum alloy, or ceramic material, or nontoxic synthetic plastics material, or any combination of these materials will be particularly useful.

As shown in the Examples below, cultured mouse fibroblasts are destroyed by silver release from metal pins having a silver electroplated finish on which an oxide layer was formed by exposure to hydrogen peroxide. Silver-plated pins lacking the oxide layer give a very weak or no cytotoxic effect.

Alloys comprising a range of silver to gold ratios were evaluated for cytocidal activity as described below in the Examples. In the interest of expediency, bacterial cultures were employed in place of fibroblast cultures. The above mentioned alloys and other implant materials are currently being evaluated using fibroblast cultures. Comparisons between the relative effects of various types of silver on a bacterial culture can be extrapolated to fibroblast cultures because silver is biologically reactive with both bacteria and fibroblasts. The E. coli strain employed is more sensitive to silver toxicity than fibroblasts, and in our experience, exposure to silver gives a zone of inhibition size about three or more times greater than the zone size obtained by exposing fibroblasts to the same material. In other words, a zone size of 2.5 mm with E. coli would correspond to a zone size of approximately 0.8 mm or less using fibroblasts. Testing is currently underway to evaluate the ability of various silver and gold alloys to inhibit fibroblast growth.

Based on the results disclosed below, we have found that alloys comprising in the range of from about 50% silver and 50% gold by composition to about 0.1% silver and about 99.9% gold by composition are suitable for use in the present invention, in that they confer to the implant the ability to prevent localized tissue growth without causing tissue irritation.

It should be appreciated that suitable alloys for use in the methods of the invention may include alloys of silver and any combination of gold, platinum, or rhodium.

The following nonlimiting examples are intended to be purely illustrative.

### EXAMPLES

### Example 1 Inhibition of Fibroblasts by Activated Silver

A 24-hr monolayer of L-929 mouse fibroblasts cells grown at 37° C. in minimal essential medium (MEM) in the presence of 5% CO₂ was overlaid with minimal essential medium (MEM) supplemented with serum, antibiotics, neutral red, and 2% agarose. Nitinol test pins were obtained from Shape Memory Applications, Inc. (Santa Clara, Calif.). Three pins were electroplated with silver (approximately 5-10 microinches thick). The silver on one of the silver plated pins was “activated” by heating in an oven at 400° F. heat for 30 minutes. The silver on a second silver plated test pin was activated by contacting it with 3% hydrogen peroxide for 30 minutes. The three silver plated pins and an unplated nitinol pin were placed on the solidified agar overlay on the fibroblast mononlayer. The cell culture was incubated at 37° C. in 5% CO₂ for 24 hours. The culture was macroscopically examined for evidence of cell deterioration. Any decolorized zone present was examined microscopically to confirm cell lysis.

Results showed that the unplated and the unactivated silver plated nitinol pins showed no zones of lysis, indicating that cytotoxicity was undetectable. The activated silver plated pins showed a mild, but significant, 1 mm zone of lysis. Additionally, suitable negative (e.g., polyethylene) and positive (e.g., latex or black rubber) controls were run, with the negative control showing no zone of lysis and the positive control showing a 5 mm zone of lysis.

### Example 2 Cytotoxic Effects of Silver Alloys and Activated Silver

Escherishia coli (ATTC 4157) was suspended in phosphate buffered saline in an amount sufficient to give a turbidity comparable to a McFarland 6 turbidity standard. A 0.1-ml aliquot of the suspension was plated and spread on standard Trypticase Soy Agar plates. The various items to be tested as described below were implanted into the agar and the bacteria were incubated at 33° C. for 24 hrs The zones of inhibition were evaluated macroscopically and measured as a relative indicator of cytocidal silver activity. Results were verified by repeating the assays at least once.

Pure silver (99.999%) wire exposed to 3% hydrogen peroxide for 30 minutes was tested as described above. Inhibition zones of about 2.5 mm were formed. Unactivated pure silver wire produced a minimal zone.

Stainless steel pins were plated with various alloys containing different ratios of silver and gold. The pins were either activated by contacting with 3% hydrogen peroxide for 30 minutes or not activated, and evaluated for cytocidal activity as described above. The results are shown in Table 1.

TABLE 1

| Alloy | Activated | Inhibition zone size |
|---|---|---|
| (% Ag:% Au) | (+/−) | (mm) |
| 100%:0% | + | 2.5 |
| 42%:58% | + | 0 |
| 25%:75% | + | 0 |
| 17%:83% | + | 0 |
| 8%:92% | + | 0 |
| 100%:0% | − | +/− |
| 42%:58% | − | 2 |
| 25%:75% | − | 2 |
| 17%:83% | − | +/− |
| 8%:92% | − | +/− |

Initially, we were surprised to learn that activated silver in alloys comprising silver and gold are not cytocidal. However, there are several factors that may contribute to this phenomenon. The oxide that formed upon activation of the silver may have functioned as a cathode conversion mask that prevented electrolyte from reaching the silver and gold corrosion cell. The amount of oxide formed may have been insufficient for detectable cytotoxicity, or the oxide formed may have been an insoluble form.

Preliminary testing comparing the relative biological effects of silver on tissue was performed indirectly using bacterial cultures. Although there is not a known direct correlation to cytotoxicity, comparisons between the relative effects of various types of silver on bacterial culture can be extrapolated to fibroblast cultures because silver is biologically reactive with both bacteria and fibroblasts. The E. coli strain employed is more sensitive to silver toxicity than fibroblasts, and exposure to silver gives a zone of inhibition size about three or more times greater than the zone size obtained by exposing fibroblasts to the same material. In other words, a zone size of 2.5 mm with E. coli roughly corresponds to a zone size of about 0.8 mm or less using fibroblasts. Testing is currently underway to evaluate the ability of various silver and gold alloys to inhibit fibroblast growth.

The invention is not limited to the exemplified embodiments, but is intended to encompass all such modifications and variations as come within the scope of the following claims.

It is claimed:

1. A method of preventing undesired postimplantation tissue growth in a human or animal subject following endoprosthetic implantation, the method comprising the steps of:
(a) selecting a suitable endoprosthetic implant, wherein the implant comprises an implant structure formed of a substantially bioinert structural material, a galvanically releasable silver component, and at least one metal more noble than silver selected from the group consisting of gold, rhodium, and platinum, wherein the silver is deposited on at least a portion of the surface of the implant structure, and wherein the more noble metal is deposited on at least a portion of the surface of the implant structure; and
(b) implanting the implant of step (a) into the subject under such conditions that a physiological electrolyte contacts the silver and the more noble metal.

2. The method of claim 1, wherein the silver component and more noble metal of step (a) are provided as an alloy.

3. The method of claim 2, wherein the concentration of silver in the alloy is effective to reduce tissue cell growth on the implant, relative to a comparable implant lacking the alloy.

4. The method of claim 2, wherein the molar ratio of silver to more noble metal in the alloy is at most about 1:1.

5. The method of claim 1, wherein the silver and the more noble metal are deposited at separate sites on the implant.

6. The method of claim 1, wherein the more noble metal is deposited as a layer on at least a portion of the implant surface, and wherein the silver is zone deposited onto a portion of the more noble metal layer.

7. A method of rendering bioactively suppressant an endoprosthetic implant comprising an implant structure formed of a substantially bioinert structural material providing mechanical integrity to the implant, a galvanically releasable silver component, and a least one metal more noble than silver selected from the group consisting of gold, platinum, and rhodium, wherein the silver and the noble metal are deposited on at least a portion of the implant structure surface, said method comprising:
(a) contacting the silver and noble metal with a physiological electrolyte.

8. The method of claim 7, wherein the silver and noble metal are provided as an alloy.

9. The method of claim 8, wherein the molar ratio of silver to more noble metal in the alloy is at most about 1:1.

10. The method of claim 7, wherein the silver and the noble metal are deposited at separate sites on the implant.

11. The method of claim 7, wherein the silver is deposited on a portion of the deposited noble metal.

12. The method of claim 7, further comprising the step of:
(c) depositing a corrosion mask on a portion of the deposited silver.

13. The method of claim 7, further comprising the step of:
(c) depositing a polymer coating permeable to a physiologic electrolye on at least a portion of the silver component.

14. A method of forming an endoprosthetic implant capable of being bioactively suppressant when contacted with a physiological electrolyte, the method comprising the steps of:
(a) providing an implant structure formed of a substantially bioinert structural material; and
(b) depositing galvanically releasable silver and a metal more noble than silver on at least a portion of the surface of the implant structure of step (a), the noble metal selected from the group consisting of gold, platinum, and rhodium.

15. The method of claim 14, wherein the silver and noble metal are provided as an alloy.

16. The method of claim 14, wherein the molar ratio of silver to the more noble metal in the alloy is at most about 1:1.

17. The method of claim 14, wherein the silver and the more noble metal are deposited at separate sites on the implant.

18. The method of claim 14, wherein the more noble metal is deposited as a layer on at least a portion of the implant surface, and wherein the silver is zone deposited onto a portion of the more noble metal layer.

19. The method of claim 14, further comprising the step of:
(c) depositing a corrosion mask on a portion of the silver.

20. The method of claim 14, further comprising the step of:
(c) depositing a polymer coating permeable to a physiologic electrolyte on at least a portion of the silver.
